(19) United States
(12) Patent Application Publication
(10) Pub. No.: US 2011/0124948 A1
(43) Pub. Date: May 26, 2011

(54) RADIOLABELED TREATMENT INFUSION SYSTEM, APPARATUS, AND METHODS OF USING THE SAME

(75) Inventor: Daniel L. Yokell, Fall River, MA (US)

(73) Assignee: Molecular Insight Pharmaceuticals, Inc.

(21) Appl. No.: 12/936,083

(22) PCT Filed: Apr. 3, 2009
(86) PCT No.: PCT/US09/39482

§ 371 Date: Jan. 10, 2011

Related U.S. Application Data
(60) Provisional application No. 61/042,592, filed on Apr. 4, 2008.

(51) Int. Cl. A61M 36/08

(52) U.S. Cl. 600/5

(57) ABSTRACT

Described herein are methods and devices for infusion of a radioactive compound, such as yttrium-90 radiolabeled somatostatin peptide or analog. A radiation shield defining a shielded cavity suitable for storing a radioactive substance includes a first aperture providing external access to the shielded cavity and a second aperture suitable for transferring a dosage vial into and out of the shielded cavity. A removable shielded plug and panel are adapted to shield respective apertures of the radiation shield. At least one dose of a radiolabeled compound stored in a vial in the radiation shield is delivered through a fluid communication channel at a rate of about 500 mL/hour. The fluid communication channel is washed after delivery, such that the process substantially reduces radiation exposure during infusion of the radiolabeled compound into a patient.

### BACKGROUND OF THE INVENTION

The present invention relates generally to the field of intravenous administration of substances to a patient and more particularly to administration of radioactive substances to the patient.

Radiopharmacology is the study and preparation of radiopharmaceuticals, i.e., radioactive pharmaceuticals. Radiopharmaceuticals are used in the field of nuclear medicine as tracers in the diagnosis and treatment of many diseases.

Radiotherapy can also be delivered through infusion (into the bloodstream) or ingestion. Examples are the infusion of metaiodobenzylguanidine (MIBG) to treat neuroblastoma, of oral iodine-131 to treat thyroid cancer or thyrotoxicosis, and of hormone-bound lutetium-177 and yttrium-90 to treat neuroendocrine tumors (peptide receptor radionuclide therapy). Another example is the injection of radioactive glass or resin microspheres into the hepatic artery to radioembolize liver tumors or liver metastases.

Radiolabeled macromolecules have also been and are being developed. Radioimmunotherapeutic agents, for example, FDA-approved Ibritumomab tiuxetan (Zevalin®), which is a monoclonal antibody anti-CD20 conjugated to a molecule of Yttrium-90, Tositumomab Iodine-131 (Bexxar®), which conjugates a molecule of Iodine-131 to the monoclonal antibody anti-CD20, were the first radioimmunotherapy agents approved for the treatment of refractory non-Hodgkin's lymphoma.

Although radiolabeled agents are being developed and are increasingly more effective at treating particular diseases and disorders, they involve certain risks, especially to health care professionals, and especially when required in large doses. Improved methods and devices are needed for the delivery of radiolabeled therapeutics.

### SUMMARY OF THE INVENTION

Described herein are infusion systems and methods for delivering a radiopharmaceutical agent to a subject, such that an administering health care professional does not get exposed to a potentially deleterious amount of radiation. The systems and methods described herein allow for combined, i.e., increased radiation doses to be delivered to the subject. The systems and methods of the present invention are useful in either diagnostic or therapeutic applications. The infusion systems of the present invention can be used to deliver any radiopharmaceutical agent that has a potentially deleterious amount of radiation, alone or in combination with one or more other substances.

One embodiment of the invention relates to a shielded enclosure suitable for reducing radiation exposure during infusion of a radioactive substance. The shielded enclosure includes a radiation shield defining a shielded cavity suitable for storing a vial containing at least one dose of a radioactive substance. The radiation shield further defines a first aperture providing external access to the shielded cavity and a second aperture suitable for transferring the vial into and out of the shielded cavity. The shielded enclosure further includes shielded plug and a shielded panel. The shielded plug is removably attachable to the radiation shield and adapted to shield the first aperture when attached thereto. Similarly, the shielded panel is also removably attachable to the radiation shield and adapted to shield the second aperture when attached thereto. The radiation shield together with the shielded plug and the shielded panel when attached, form a substantially continuous shielded cavity, providing radiation shielding suitable for reducing radiation exposure during infusion of the radioactive substance from the vial to a patient.

In some embodiments, the radiation shield includes more than one different shielding layers. The shielded plug and shielded panel are also configured, when attached to the radiation shield, to preserve continuity of the more than one different shielding layers about the substantially continuous shielded cavity. In some embodiments, each of the more than one shielding layers is formed from a respective material selected from a group of materials consisting of: metals; aluminum; lead; steel; stainless steel; tungsten; titanium; metal alloys; leaded glass; polymers; polycarbonate materials; solids formed from synthetic resins; and wood. In some embodiments, each of the more than one shielding layers is formed from one or more non-porous materials selected from, e.g., but not limited to, metals, metal alloys, amorphous materials, such as glass, and hard plastic, or derivatives thereof. In some embodiments, the radiation shield includes an inner layer of polycarbonate material and an outer layer of metal, such as aluminum. In some embodiments, the radiation shield includes an attachment element allowing it to be suspended, for example, from an intravenous (IV) pole. The vial stored within the shielded cavity contains at least one dose of a radioactive substance and has an access port substantially aligned with the first aperture when stored within the shielded cavity. The radioactive substance can be a yttrium-90 radiolabeled somatostatin peptide or analog.

Another embodiment of the invention relates to a process for administering a radiolabeled compound to a patient. The process includes placing a reservoir containing at least one dose of a radioactive compound in a shielded enclosure having a fluid access port. A fluid communication channel is provided between the reservoir and a patient. At least one dose of the radiolabeled compound is delivered through the fluid communication channel at a rate of about 500 mL/hour. The fluid communication channel is washed after delivery of the radiolabeled compound, such that the process substantially reduces radiation exposure during infusion of the radiolabeled compound into a patient.

In some embodiments, a saline solution is flushed through the fluid communication channel to wash the fluid communication channel. In some embodiments, the shielded enclosure includes an interior polycarbonate layer and an exterior aluminum layer. In some embodiments, the radiolabeled substance is yttrium-90 radiolabeled somatostatin peptide or analog. In some embodiments, a non-radiolabeled compound is also delivered through the fluid communication channel at a rate of about 500 mL/hour. Delivery of the radiolabeled compound and the non-radiolabeled compound can occur in succession.

Another embodiment of the invention relates to an intravenous injection apparatus including a first reservoir storing a first non-radioactive compound, a first fluid line in fluid communication between the first reservoir and a patient-side needle. The injection apparatus also includes a second reservoir storing a saline solution, a second fluid line in fluid communication with the patient-side needle, and a vial shield surrounding a vial containing a radioactive compound. The vial is in fluid communication with the second fluid line, such that the apparatus is configured to inject a dose of radioactive compound into a living subject operably coupled to the second end of the fluid line.

In some embodiments, the vial shield comprises a substantially continuous aluminum shielding layer and a substantially continuous polycarbonate material shielding layer; the vial shield further including an access aperture providing access through the shielding layers. In some embodiments, the radioactive compound is a radioconjugate including an yttrium-90 radiolabeled somatostatin peptide or analog. In some embodiments, the non-radioactive compound includes a diluted nutrient preparation containing amino acids. In some embodiments, the intravenous injection apparatus further includes a dual channel infusion pump. A first channel of the pump is adapted for infusing a fluid through the first fluid line and a second channel of the pump adapted for infusing a fluid through the second fluid line.

Yet another embodiment of the invention relates to a process for reducing radiation exposure during infusion of a radioactive compound into a patient. The process includes storing a vial containing at least one dose of a radioactive zo compound in a shielded enclosure having an aperture blocked by a shielded access plug. The shielded access plug is removed from shielded enclosure thereby exposing the aperture. An intravenous (IV) fluid line is coupled between the vial containing the at least one dose of a radioactive compound and the patient, the coupling occurring through the exposed aperture. At least a portion of the at least one dose of a radioactive compound is infused into the patient through the IV fluid line.

In some embodiments, the radioactive compound is a radioconjugate including an yttrium-90 radiolabeled somatostatin peptide or analog. In some embodiments, a non-radioactive compound is also infused into the patient through at least a patient proximal portion of the IV fluid line. In some embodiments infusing both radioactive and non-radioactive compounds, the non-radioactive compound is a diluted nutrient preparation containing amino acids and the radioactive compound is a radioconjugate comprising an yttrium-90 radiolabeled somatostatin peptide or analog, each being infused alternately into the patient through at least a portion of the IV fluid line. In some embodiments, the shielded enclosure containing at least one dose of a radioactive compound is suspended from an IV pole.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic representation of an embodiment of a infusion system configured for intravenously administering a radioactive substance.

FIG. 2 is an exploded perspective view of an embodiment of a vial shield.

FIGS. 3A and 3B are side and bottom views, respectively, of the exemplary open-ended radiation-shielded vessel shown in FIG. 2.

FIGS. 4A and 4B are top and side views, respectively, of the exemplary radiation-shielded plug shown in FIG. 2.

FIGS. 5A and 5B are top and side views, respectively, of the exemplary removable radiation-shielded cover shown in FIG. 2.

FIG. 5C is a sectional view along A-A of the embodiment of the removable radiation-shielded cover illustrated in FIG. 5A.

FIG. 6 is a flow diagram of an embodiment of a process for intravenously administering a radioactive substance.

### DETAILED DESCRIPTION OF THE PREFERRED EMBODIMENTS

The invention pertains to systems and processes for administering a radioactive substance to a patient. The systems and processes of the invention are useful in both diagnostic (e.g., in vivo imaging) and therapeutic applications. The radioactive substance may be formulated as a radiolabeled imaging agent or a radiolabeled therapeutic agent. In one embodiment the radioactive substance is a radiolabeled imaging agent, such as such as an yttrium-90 radiolabeled somatostatin peptide or analog. Alternatively or in addition, the radioactive substance is formulated or combined with one or more other substances to form a radiotherapeutic substance. A suitable delivery system includes, for example, a pump to deliver the radioactive substance at a desired infusion rate. For example, the pump can be configured to infuse a peptide or analog at a rate of, for example, about 500 mL/hour. The systems and processes optionally include provisions for washing or otherwise flushing at least that portion of the intravenous (IV) tubing exposed to the radioactive substance after delivery of the radioactive substances, e.g., radiolabeled peptide or analog.

A radioconjugate consisting of the octreotide derivative edotreotide labeled with yttrium 90 (Y-90) has potential radiotherapeutic uses. Similar to octreotide, yttrium Y-90 edotreotide binds to somatostatin receptors (SSTRs), especially type 2 receptors, present on the cell membranes of many types of neuroendocrine tumor cells, delivering tissue-specific, beta-emitting nuclide Y-90-mediated cytotoxicity to SSTR-positive cells. Ythium Y-90 edotreotide is produced by substituting tyrosine for phenylalanine at the 3 position of the somatostatin analogue octreotide and chelating the substituted octreotide to Y-90 via dodecanetetraacetic acid (DOTA).

Onalta® (Molecular Insight Pharmaceuticals, Cambridge, Mass. USA) is a radiotherapeutic product for the treatment of cancer. Formerly known as OctreoTher, Onalta® is the brand name for edotreotide, an yttrium-90 (Y-90) radiolabeled somatostatin peptide. Somatostatin is a hormone distributed throughout the body that acts as a regulator of endocrine and nervous system function by inhibiting the secretion of several other hormones such as growth hormones, insulin and gastrin. Onalta® is useful for the radiotherapeutic treatment of metastatic carcinoid and pancreatic neuroendocrine cancer in patients whose symptoms are not controlled by conventional somatostatin analog therapy. Somatostatin analog therapy (or octreotide or sandostatin) is used to alleviate the symptoms associated with carcinoid syndrome.

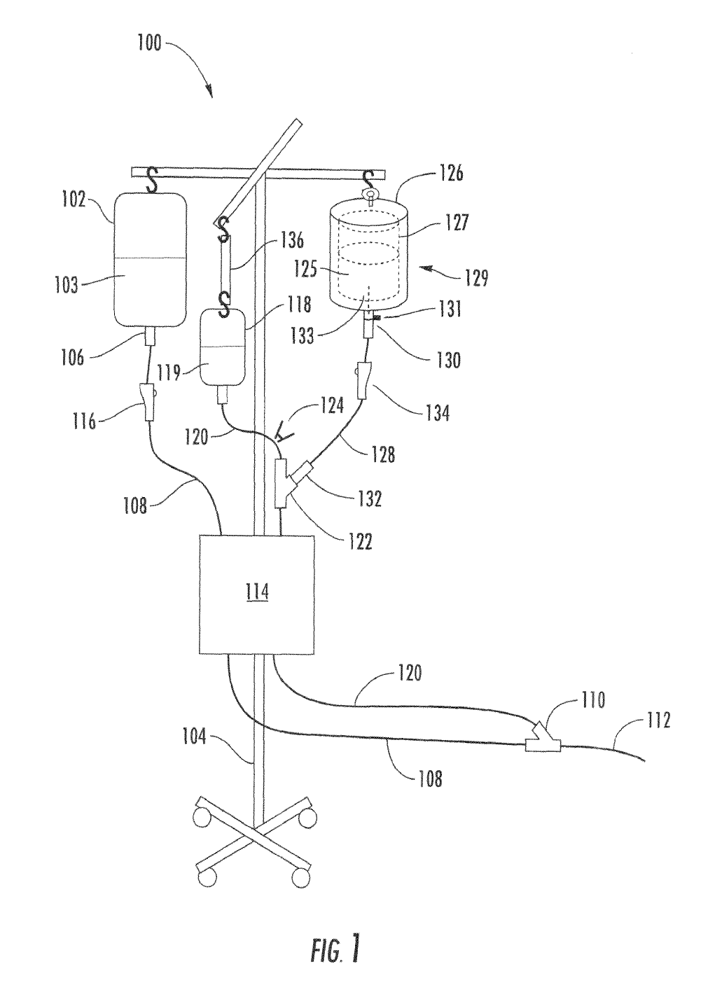

A schematic representation of an embodiment of a infusion system configured for intravenously administering a radioactive substance is illustrated in FIG. 1. An IV setup 100 includes a primary IV supply or bag 102 suspended from a top portion of an IV pole 104. The primary IV bag 102 includes a drip chamber 106 coupled to a distal end of primary IV tubing 108. The proximal end of the primary IV tubing 108 terminates in a respective port of a fluid junction 110, sometimes referred to as a “Y-site.” An IV tubing extension 112 is coupled between a respective port of the Y-site 110 and a patient (not shown). The primary IV tubing 108 is routed through a first channel of a dual-channel infusion pump 114. The infusion pump 114 is positioned between the primary IV bag 102 and the Y-site 110 and configured to infuse a first non-radioactive substance from the primary IV bag 102 into the patient. A flow-control valve 116, such as a roller valve, is positioned between the infusion pump 114 and the drip chamber 106 of the primary IV bag 102, which can be used to establish a desired flow rate of the non-radioactive substance.

The IV setup 100 also includes a secondary IV supply or bag 118 suspended from the top portion of the IV pole 104. The secondary IV bag 118 is coupled to a distal end of secondary IV tubing 120. The proximal end of the secondary IV tubing 120 terminates in a respective port of the Y-site 110. The secondary IV tubing 120 is routed through a second channel of the dual-channel infusion pump 114. The infusion pump 114 is similarly positioned between the secondary IV bag 118 and the Y-site 110 and configured to infuse a second non-radioactive substance from the secondary IV bag 118 into the patient. An access port 122, sometimes referred to as an injection port is positioned along the secondary IV tubing 120, between the infusion pump 114 and the secondary IV bag 118. The access port 122 provides a location for fluid access to a fluid channel of the secondary IV tubing 120. In some embodiments, a flow control device, such as a slide clamp or “A clamp” 124 is positioned along the secondary IV tubing 120, between the access port 122 and the secondary IV bag 118, which can be used to interrupt or otherwise control flow of fluid from the secondary IV bag. Alternatively or in addition, a flow-control valve (not shown), such as a roller valve, is positioned between the access port 122 and the secondary IV bag 118.

The IV setup 100 further includes shielded container, such as a vial shield 126 suspended from the top portion of the IV pole 104. The vial shield 126 includes an interior shielded region sized and shaped to accommodate therein a patient dose vial 127. Patient dose vials 127 intended for use in the vial shield 126, generally contain a radioactive substance. Each patient dose vial 127 also includes at least one fluid access port. For example, the fluid access port can be a piercable region, such as the vial septum of a common dose vial. When the patient dose vial 127 is positioned within the vial shield 126, the vial septum, is aligned with a resealable vial shield aperture. In some embodiments, the patient dose vial 127 includes a vent allowing for pressure equalization of an interior of the patient dose vial 127 and the surrounding environment. The vial shield 126 allows for safe handling and administration of radioactive substances, the particular shielding properties being designed to greatly reduce exposure to non-patient individuals from radioactive material contained within the patient dose vials 127.

The shielded patient dose vial 129 is coupled to a distal end of a length of auxiliary IV tubing 128. An extraction apparatus 133 is used to provide fluid communication between a patient dose contained in the patient dose vial 129 and the auxiliary IV tubing 128. The extraction apparatus can use suction or vacuum action. In some embodiments, the extraction apparatus is a piercing cannula, such as a hypodermic needle or IV spike 133. A drip chamber 130 is typically positioned between the distal end of the auxiliary IV tubing 128 and the resealable aperture of the shielded patient dose vial 129. The proximal end of the auxiliary IV tubing 128 terminates in a fluid connector 132 adapted for fluid communication through the access port 122, thereby providing fluid access between the auxiliary IV tubing 128 and the secondary IV tubing 120. A flow-control valve 134, such as a roller valve, is positioned between the fluid connector 132 and the drip chamber 130 of the infusion shielded patient dose vial 129, which can be used to establish a desired flow rate of the radioactive substance. The secondary IV bag 118 is suspended from the top of the IV pole 104 by way of an extension 136, such the secondary IV bag 118 is relatively lower than the shielded patient dose vial 129.

The secondary IV bag 118 is configured to infuse a second non-radioactive substance from the secondary IV bag 118, through the Y-site 110, into the IV tubing extension 112 and ultimately into the patient. An access port 122, sometimes referred to as an injection port, is positioned along the secondary IV tubing 120, between the infusion pump 114 and the secondary IV bag 118. The access port 122 provides a location for fluid access to a fluid channel of the secondary IV tubing 120. In some embodiments, a flow control device, such as a slide clamp or “A clamp” 124 is positioned along the secondary IV tubing 120, between the access port 122 and the secondary IV bag 118. Alternatively or in addition, a flow-control valve (not shown), such as a roller valve, is positioned between the access port 122 and the secondary IV bag 118. In some embodiments, one or more of the IV tubing extension and at least proximal portion of the secondary IV tubing are radiation shielded.

In operation, the IV setup 100 allows a radioactive solution to be infused from the patient dose vial 127 to the patient through an IV access site. The access site can include without restriction an antecubital or equivalent vein. Generally, any IV suitable access site, such as a central catheter, can also be used. A multiport fluid coupling, such as the Y-site 110, allows more than one IV sources to be injected into a patient through the same IV access site. The primary IV bag 102 is hung from the IV stand 104, and spiked using an infusion pump primary set 108. An infusion set typically includes a spike, a drip chamber, and a plastic high pressure tube, with the spike configured to pierce an IV fluid reservoir, such as the primary IV bag 102. The primary IV tubing is then primed, to remove air. In some embodiments, a check valve is included along the IV tubing. Alternatively or in addition, the infusion set includes a vent. For example, a vent 131 can be provided on the IV spike 133, or top portion of the drip chamber 130 to provide venting when necessary. Since the patient dose vial 127 may have rigid or semi-rigid walls, equalization of the pressure across the walls is required to allow fluid transfer with the shielded patient dose vial 129. In such instances, a separate vent can be provided on the patient dose vial 129 itself.

The primary IV tubing 108 is inserted into the primary channel of a dual channel infusion pump, with a patient end of the tubing attached to a first port of the Y-site 110. In an exemplary embodiment, the primary IV bag 102 includes a non-radioactive solution 103, such as a nutrient preparation. For example, the primary IV bag 102 includes about 1000 mL of a 7% nutrient preparation 103 containing amino acids, such as Aminosyn® II amino acid solution (Aminosyn is a registered trademark of Hospira, Inc. of Lake Forest, Ill.).

An infusion rate of fluid flowing from the primary IV bag 102 through the primary IV tubing 108 is set at or otherwise adjusted to a preferred infusion rate using generally well understood techniques for adjusting infusion rates. For example, an infusion rate of the 7% Aminosyn® II amino acid is set at a recommended infusion rate of about 500 mL per hour. The first channel of the dual channel infusion pump 114 is adjusted to begin the infusion of Aminosyn® through the primary line and to maintain infusion for a primary infusion interval, e.g., for at least 30 minutes.

The secondary IV bag 118 is hung from the IV stand 104, the secondary IV bag 118 is also spiked using an infusion pump secondary set. The secondary tubing 120 is then primed to substantially remove any air within the line. For example, the secondary IV bag 118 includes about a 100 mL of 0.9% sodium chloride solution 119 for injection. The secondary IV tubing 120 is inserted into the secondary channel (Channel 2) on the dual channel infusion pump 114 with its patient end attached to a second port of the Y-Site 110.

Infusion of the first non-radioactive substance, e.g., the 7% Aminosyn® II amino acid solution infusion, is commenced for primary infusion interval and then paused. An infusion rate for the secondary IV bag 118 is set at a respective infusion rate using generally well understood techniques for setting or otherwise adjusting the rate. For example, the infusion rate of the 0.9% Sodium Chloride Solution (Channel 1) is set at about 500 mL per hour. Infusion of the secondary IV bag contents 119 is initiated and allowed to run for a relatively brief interval, e.g., for a few minutes to ensure that flow from the secondary IV bag is acceptable (e.g., desired flow rate).

The shielded patient dose vial 129 includes a vial shield 126 having an interior shielded cavity containing a patient dose vial 127. The patient dose vial 127, in turn, includes a radioactive substance 125 to be administered to the patient. The shielded patient dose vial 129 is hung from the IV stand 104. Using an extension hanger 136, the secondary IV bag 118 is lowered, such that the secondary IV bag 118, e.g., containing the 0.9% sodium chloride, is positioned below the level of the patient dose vial 127.

The secondary set fluid connector 132 attached to a proximal end of the auxiliary IV tubing 128 line is insert the connector 122 positioned along the secondary IV tubing 120, at a height above the pump 114. A flow control device, such as a roll clamp 134 is positioned along the auxiliary IV tubing and adjusted allow to allow saline solution from the secondary IV bag to prime the auxiliary IV tubing 128. The roll clamp 134 is closed once the saline has reached the drip chamber 130 of the auxiliary tubing.

The patient dose vials 127 containing the radioactive substance 125, e.g., Onalta® (Y-90 Edotreotide), is inverted and placed within the infusion shield 126. An access plug is removed from a bottom of the infusion shield 126 providing access to an injection port of the patient dose vial 127 contained therein. The patient dose vial 127 is then spiked inside the infusion shield 126 with the auxiliary IV set spike 133. The shielded patient dose vial 129 is hung from the IV stand 104 and a vent cap 131 opened. The arrangement of the secondary IV 118 bag containing the sodium chloride solution 119 and the patient dose vial 127 positioned and attached as described herein is sometimes referred to as a “piggy back” arrangement. When the secondary IV bag 118 and the shielded patient dose vial 129 are connected and positioned as described, the patient dose 125 will infuse first (higher pressure), and when depleted, automatically be followed by infusion of the secondary IV bag contents 119 in a substantially uninterrupted manner.

The infusion pump is suitably configured, e.g., using a piggy-back setting when available, to set or otherwise adjust an infusion rate of the radioactive substance 125, e.g., Onalta® (Y-90 Edotreotide) at the desired infusion rate. For the exemplary Onalta® (Y-90 Edotreotide), the fill volume in the patient dose vial is about 86 mL, and a recommended infusion rate is about 500 mL per hour. The infusion of Onalta® (Y-90 Edotreotide) can be adjusted to occur over 10 minutes at the recommended rate. Infusion of the radioactive substance can be adjusted by the roll clamp on the auxiliary IV tubing line. To begin infusion, the roll clamp on the auxiliary IV tubing line is released.

Once infusion of the radioactive substance 125, e.g., Onalta® (Y-90 Edotreotide), has finished and the saline 119 has restarted, flow of the saline 119 can be interrupted using a clamp, such as the A-clamp 124 positioned along the secondary line 120 and above the injection site. The saline line is clamped above a check valve (not shown), when provided, to administer any remaining Onalta® in the auxiliary line—infusion of saline 119 stops, while infusion of any residual radioactive substance empties at least the auxiliary IV line 128. The clamp 124 is released once the contents of the auxiliary IV line 128 have been administered. Infusion of the saline can be restarted to infuse any residual Onalta® from the patient end of the secondary IV tubing 120, and avoid any mixing of the primary IV contents 103 with the radioactive drug product 125.

The radioactive material 125 provided in the shielded patient dose vial 129 can be an imaging agent, such as a radiopharmaceutical composition for in-vivo imaging. Exemplary radiopharmaceutical compositions include Zemiva® (iodofiltic acid 1123) used in the a detection and management of cardiac ischemia by imaging metabolic changes in the heart, and Trofex® used in the detection monitoring or therapy of prostate cancer via binding to prostate-specific membrane anginen (PSMA). Alternatively or in addition, the radioactive material can be a therapeutic material, such as a radiopharmaceutical composition for treating cancer. Exemplary radiotherapeutic materials include Azedra® (Ultratrace® iobenguane 1131) used in the treatment of neuroendocrine tumors using a tumor's norepinephrine uptake mechanism, Solazed® (1-131 labeled benzamide) used in the treatment of metastatic melanoma based on melanin-binding small molecule, and Onalta® (yttrium-90 radiolabeled somatostatin peptide analog, such as an edotreotide) used in the treatment of carcinoid tumors using receptor-based radiotherapuetic. Zemiva®, Trofex®, Azedra®, Solazed®, Ultratrace® and Onalta® are registered trademarks of Molecular Insight Pharmaceuticals, Inc. of Cambridge, Mass.

In some embodiments, the radioactive material 125 provided in the shielded patient dose vial 129 can include a radiopharmacological agent labeled with an isotope selected from the group consisting of one or more of: Technetium-99m (technetium-99m), Iodine-123, Iodine-125 and Iodine-131, Thallium-201, Gallium-67, Yttrium-90, Samarium-153, Strontium-89, Phosphorous-32, Rhenium-186, Lutetium-177, Fluorine-18 and Indium-111 and/or an isotope as summarized in Table 1 below.

TABLE 1

| Isotope | Exemplary Diagnostic/Therapeutic/Medical Use |
|---|---|
| Molybdenum-99 | Used as the ‘parent’ in a generator to produce |
| | technetium-99m. |
| Technetium-99m | Used in to image the skeleton and heart muscle |
| | in particular, but also for brain, thyroid, lungs |
| | (perfusion and ventilation), liver, spleen, kidney |
| | (structure and filtration rate), gall bladder, bone |
| | marrow, salivary and lacrimal glands, heart |
| | blood pool, infection and numerous specialized |
| | medical studies. |
| Bismuth-213 | Used for TAT. |
| Chromium-51 | Used to label red blood cells and quantify gastro- |
| | intestinal protein loss. |
| Cobalt-60 | Formerly used for external beam radiotherapy. |
| Copper-64 | Used to study genetic diseases affecting copper |
| | metabolism, such as Wilson's and Menke's |
| | diseases. |
| Dysprosium-165 | Used as an aggregated hydroxide for |
| | synovectomy treatment of arthritis. |
| Erbium-169 | Use for relieving arthritis pain in synovial joints. |
| Holmium-166 | Being developed for diagnosis and treatment of |
| | liver tumors. |
| Iodine-125 | Used in cancer brachytherapy (prostate and |
| | brain), also diagnostically to evaluate the filtration |
| | rate of kidneys and to diagnose deep vein |
| | thrombosis in the leg. It is also widely used in radio- |
| | immuno-assays to show the presence of hormones |
| | in tiny quantities. |
| Iodine-131 | Widely used in treating thyroid cancer and in |
| | imaging the thyroid; also in diagnosis of abnormal |
| | liver function, renal (kidney) blood flow and urinary |
| | tract obstruction. A strong gamma emitter, but used |
| | for beta therapy. |
| Iridium-192 | Supplied in wire form for use as an internal |
| | radiotherapy source for cancer treatment (used then |
| | removed). |
| Iron-59 | Used in studies of iron metabolism in the spleen. |
| Lutetium-177 | Lu-177 is increasingly important as it emits just |
| | enough gamma for imaging while the beta radiation |
| | does the therapy on small (e.g., endocrine) tumors. |
| | Its half-life is long enough to allow sophisticated |
| | preparation for use. |
| Palladium-103 | Used to make brachytherapy permanent implant |
| | seeds for early stage prostate cancer. |
| Phosphorus-32 | Used in the treatment of polycythemia vera (excess |
| | red blood cells). Beta emitter. |
| Potassium-42 | Used for the determination of exchangeable |
| | potassium in coronary blood flow. |
| Rhenium-186 | Used for pain relief in bone cancer. Beta emitter |
| | with weak gamma for imaging. |
| Rhenium-188 | Used to beta irradiate coronary arteries from an |
| | angioplasty balloon. |
| Samarium-153 | Sm-153 is very effective in relieving the pain of |
| | secondary cancers lodged in the bone. Also very |
| | effective for prostate and breast cancer. Beta emitter. |
| Selenium-75 | Used in the form of seleno-methionine to study the |
| | production of digestive enzymes. |
| Sodium-24 | For studies of electrolytes within the body. |
| Strontium-89 | Very effective in reducing the pain of prostate and |
| | bone cancer. Beta emitter. |
| Xenon-133 | Used for pulmonary (lung) ventilation studies. |
| Ytterbium-169 | Used for cerebrospinal fluid studies in the brain. |
| Yttrium-90 | Used for cancer brachytherapy and as silicate |
| | colloid for the relieving the pain of arthritis in larger |
| | synovial joints. Pure beta emitter. Radioisotopes of |
| | cesium, gold and ruthenium are also used in |
| | brachytherapy. |
| Carbon-11, | These are positron emitters used in PET for |
| Nitrogen-13, | studying brain physiology and pathology, in |
| Oxygen-15, | particular for localizing epileptic focus, and in |
| Fluorine-18 | dementia, psychiatry and neuropharmacology |
| | studies. They also have a significant role in |
| | cardiology. F-18 in FDG has become very important |
| | in detection of cancers and the monitoring of |
| | progress in their treatment, using PET. |
| Cobalt-57 | Used as a marker to estimate organ size and for in- |
| | vitro diagnostic kits. |
| Gallium-67 | Used for tumor imaging and localization of |
| | inflammatory lesions (infections). |
| Indium-111 | Used for specialist diagnostic studies, e.g., brain |
| | studies, infection and colon transit studies. |
| Iodine-123 | Increasingly used for diagnosis of thyroid function, |
| | it is a gamma emitter without the beta radiation of |
| | I-131. |
| Krypton-81m from | Kr-81m gas can yield functional images of |
| Rubidium-81 | pulmonary ventilation, e.g., in asthmatic patients, |
| | and for the early diagnosis of lung diseases and |
| | function. |
| Rubidium-82 | Convenient PET agent in myocardial perfusion |
| | imaging. |
| Strontium-92 | Used as the ‘parent’ in a generator to produce Rb-82. |
| Thallium-201 | Used for diagnosis of coronary artery disease other |
| | heart conditions such as heart muscle death and for |
| | location of low-grade lymphomas. |

Alternatively or in addition, the radioactive material 125 can be selected from the group consisting of one or more of Bexxar® (Iodine 1-131 Tositumomab), Zevalin®(Yttrium Y-90 Ibritumomab Tiuxetan), Quadramet® (Samarium Sm-153 Lexidronam), Strontium-89 chloride, Phosphorous-32, Rhenium-186 hydroxyethlidene, Samarium-153 lexidronam, I-131. Bexxar® is a registered trademark of SmithKline Beecham Corporation of Philadelphia, Pa. Zevalin® is a registered trademark of Cell Therapeutics, Inc. of Seattle, Wash., and Quadramet® is a registered trademark of Cytogen Corporation of Princeton, N.J.

More than one vial of radioactive substance can be administered, if necessary, to fulfill the total patient dose. When administering two or more patient dose vials, the same piggyback arrangement can be used. Namely, once the contents of the first patient dose vial 127 have been emptied, a second patient dose vial 127′, e.g., containing a second dose of Onalta® (Y-90 Edotreotide), is inverted and spiked after being suitably positioned within the infusion shield 126. The shielded patient dose vial 129 the second patient dose vial 127′ is hung from the IV stand 104 and the auxiliary IV tubing 128 re-primed. Radioactive contents 125′ of the second patient dose vial 127′ can be infused at the same rate of 500 mL per hour, or at a different rate, if necessary. Once the contents of the patient dose vial(s) have been infused, the auxiliary and secondary lines 128, 120 can be flushed with the zo remainder of the 0.9% sodium chloride bag 118. Once the secondary IV tubing line 120 has been flushed with the remainder of the 0.9% sodium chloride bag 118, infusion of contents of the primary IV bag 102, e.g., the Aminosyn® Amino Acid 103, is resumed at a respective infusion rate. The respective infusion rate of the Aminosyn® Amino Acid 103 may be the same as the previous rate of about 500 mL per hour, or at a different rate.

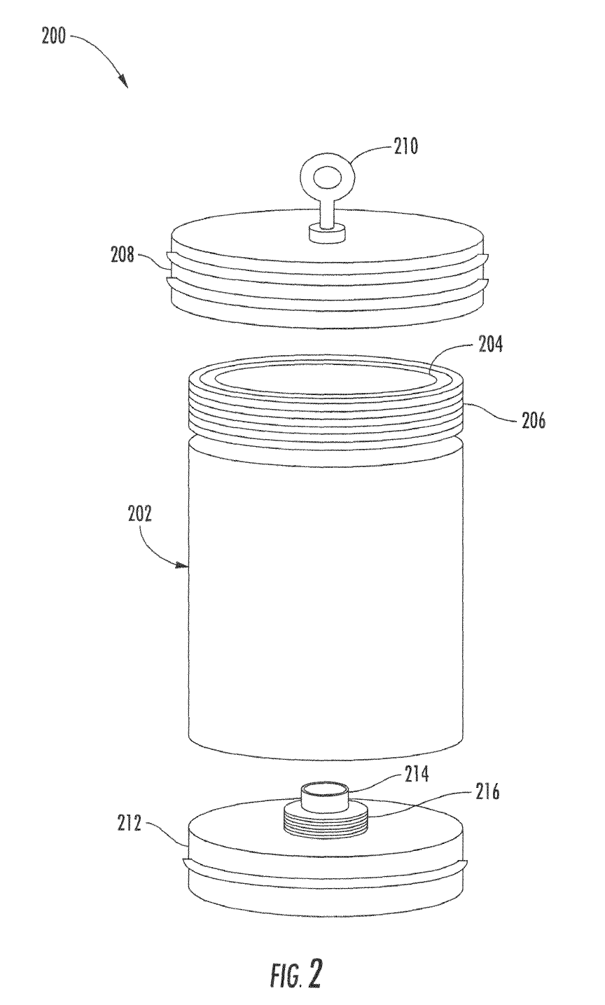

An exploded perspective view of an embodiment of a vial shield 200 is illustrated in FIG. 2. The infusion shield 200 includes an open-ended shielded vessel 202 having a relatively wide opening 204 for removal and replacement of patient dose vials 127 (FIG. 1). This relatively wide opening 204 can be placed at one end of the shielded vessel 202, such as the top end as illustrated. In the exemplary embodiment, the infusion shield 200 has a generally cylindrical shape, defining a substantially cylindrical interior shielded chamber. In some embodiments, the dimensions of the interior shielded chamber are selected according to dimensions and shape of patient dose vials to be stored therein. For example, the dimensions can be selected to allow for supportively storing the patient dose vial with little or no gaps to ensure a snug fit. Other container shapes are possible, such as polygons, ellipsoids, etc.

The infusion shield 200 includes a shielded lid 208 configures for removable attachment to the relatively wide opening 204 of the shielded vessel 202. Removal of the shielded lid 208 allows for access to the interior shielded chamber of the open-ended vessel 202 through the relatively wide opening 204, for example, to insert and remove patient dose vials containing radioactive material. The shielded vessel 202 includes an attachment feature to facilitate removable attachment of the shielded lid 208 from the shielded vessel 202. For example, the attachment feature includes a thread 206, suitably positioned with respect to the relatively wide opening 204, and the shielded lid 208 includes a complementary attachment feature, such as a complementary thread to allow for removable attachment of the shielded lid 208 from the shielded vessel 202. In some embodiments, the shielded lid 208 includes an attachment mechanism to facilitate removable attachment of the infusion shield 200 to an IV stand. The attachment mechanism can include an eyelet 210, or other suitable anchor, hook, handle attached to support the infusion shield 200 in the upright position during use. As illustrated, the eyelet 210 is attached at the center of a top exposed surface of the shielded lid 208.

The infusion shield 200 further includes a removable shielded plug 212 allowing controlled access to an interior region of the infusion shield 200 when removed. For example, removal of the shielded plug 212 exposes a relatively small aperture providing an access channel to a patient dose vial stored therein. Such access can be obtained by a spike of an IV tubing set, allowing fluid communication via the IV tubing to the patient dose vial stored therein. In the exemplary embodiment, the removable shielded plug 212 includes an inner shielded bung 214 configured for insertion into a receptacle provided along the bottom surface of the open-ended shielded vessel 202. A suitable removable fastening arrangement, e.g., a threaded arrangement, is used for removable attachment of the shielded plug 212 from the infusion shield 200. The threaded arrangement can also be used to engage a portion of an interconnected IV set, such as a Luer lock style threaded arrangement.

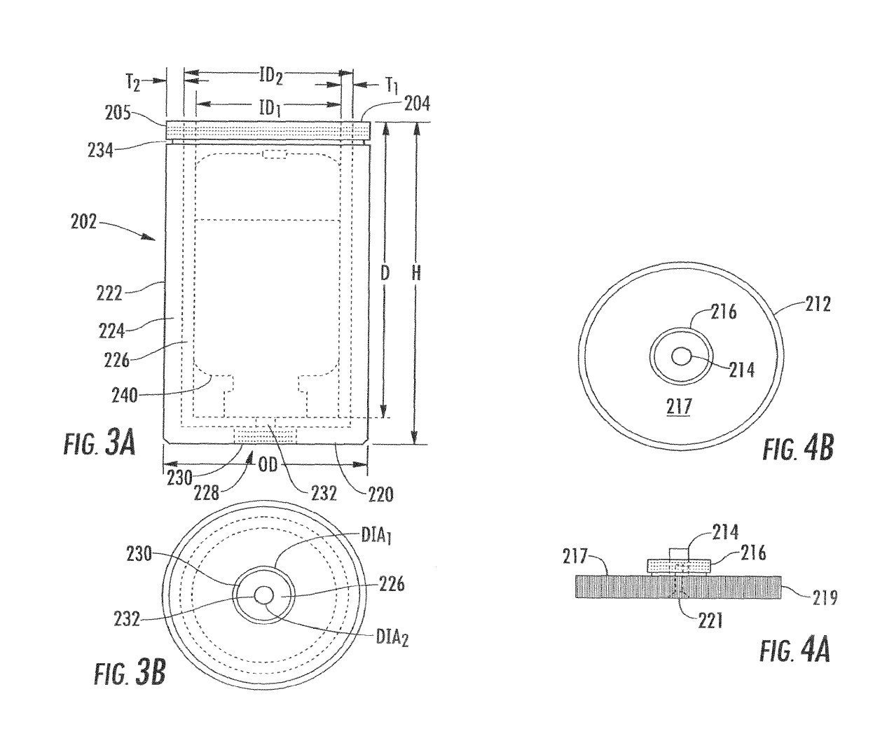

FIGS. 3A and 3B are side and bottom views, respectively, of the exemplary open-ended radiation-shielded vessel 202 shown in FIG. 2. The open-ended vessel 202 includes a radiation-shielded bottom wall 220 disposed opposite the relatively wide open end 204. An elongated radiation-shielded side wall 222 extends between the bottom wall 222 and the open end 224. The bottom and side walls 220, 222 are suitably formed to provide an acceptable level of radiation shielding for patients and clinicians to patient dosage vials including radioactive substances, such as Onalta® (Y-90 Edotreotide). Radiation materials suitable for shielding include metals, such as aluminum, lead, steel, stainless steel, tungsten, titanium, metal alloys, leaded glass, polymers, Lexan® (polycarbonate material), Plexiglas®, Lucite® (synthetic resin materials), and even wood, provided alone or in combination. Plexiglas® is a registered trademark of Arkema France Corp. of Colombes, France. Lexan® is a registered trademark of Sabic Innovative Plastics IP B.V. Company of Pittsfield, Mass. and Lucite® is a registered trademark of Lucite International, Inc. of Cordova, Tenn. In the illustrated embodiment, the bottom and side walls 220, 222 are formed using multiple layers of different materials. In particular, the walls 220, 222 include an outer layer of a metal, such as aluminum 224 and an inner layer of a glass or polymer, such as Lexan® 226. The inner and outer layers 226, 224 extend substantially uninterrupted except for the open end 204 and an access port 228 centrally located in the bottom wall 220. The access port 228 includes a threaded aperture 230 extending through the outer aluminum layer 224 of the bottom wall 220 and a coaxial aperture 232 extending through the inner, Lexan layer 226 of the bottom wall 220.

The shape and dimensions of the infusion shield 200 can be selected depending upon factors, such as patient dose vial size and shape. An exemplary patient dose vial 240 is illustrated in phantom, stored within the cavity of the shield. The patient dose vial 240 is positioned such that an access port, e.g., a septum, is positioned adjacent to the coaxial aperture 232. For the exemplary 86 mL patient dose of Onalta® (Y-90 Edotreotide), the external height ‘H’ of the side wall 222 measured from the outer surface of the bottom wall 220 to the open end 204 is about 4.4 inches. The inner height ‘D’ of the side wall 222 measured from the inner surface of the bottom wall 220 to the open end 204 is about 3.89 inches. The outer diameter ‘OD’ of the open-ended vessel 202 is about 2.98 inches. The inner diameter ‘ID₁’ of the vessel chamber is about 2.07 inches at the open end 204. The inner diameter ‘ID₂’ of the aluminum layer 224 is about 2.468 (−0.003 in., +0.002 in.).

FIGS. 4A and 4B are top and side views, respectively, of the exemplary radiation-shielded plug shown in FIG. 2. The IV port shielded plug 212 includes a support member 217, such as the flat disk shaped support member 217 illustrated, onto which two or more bung elements 216, 214 are securely attached. Each bung element 216, 214 is composed of a respective radiation shielding material, each configured to complete a respective portion of the shield of the open-ended vessel 202 when the plug 212 is inserted into the IV access aperture 228. In the illustrative example, an outer bung element 216 is a disk shaped plug of metallic shield material, such as aluminum, sized and shaped to fit snugly into the aluminum aperture in the outer shield layer 224 of the IV access aperture 228. An inner bung element 214 positioned along a top surface of the lower bung element 216 is a cylindrical shaped plug of polymer shield material, such as Lexan®. The inner bung element 214 is sized and shaped to fit snugly into the Lexan aperture in the inner shield layer 226 of the IV access aperture 228.

Each of the bung elements 214, 216 are securely attached to the support member 217. A screw, such as the flathead screw 221 shown in FIG. 4A can be used to fasten the various elements 214, 216, 217 of the shielded plug 212 together as shown. Alternatively or in addition, one or more other fastening means may be employed, such as chemical glues and epoxies, rivets, staples, welds, etc.

The IV port shielded plug 212 further includes a fastening feature to allow removable attachment of the plug 212 to the open-ended shielded vessel 202. In the exemplary embodiment, the outer bung element 216 includes a peripheral thread around at least a portion of the perimeter of the disk. The thread is sized and shaped according to a complementary thread provided on 230 along the outer shield of the IV access aperture 228. Thus, the shielded plug 212 can be fastened to the bottom of the shielded vessel 202 by aligning the inner bung element 214 with the IV access aperture 228, inserting the shielded plug 212 partially into the IV access aperture 228, and engaging the threads of the outer bung element 216 with threads along the outer shield of the IV access aperture 228. A frictional surface, such as a knurl 219 can be provided along at least a portion of the outer perimeter of the supporting member 217, to form a grip for a thumbwheel, allowing for easy insertion and removal of the plug 212. Other fastening features are possible, such as threads along the upper bung element 214, threads along the supporting member to engage complementary threads along the bottom wall 220 of the shielded vessel 202, and other fastening members, such as screws, clips, etc. When inserted into the IV aperture 228 and secured to the shielded vessel 202, the shielded plug 212 substantially fills the IV aperture 228 in such a manner that the corresponding portions of the inner and outer shield layers 226, 224 are substantially continuous. Thus, in this example, the inner shield 226 of the bottom wall 220 is substantially continuous as is the outer shield 224.

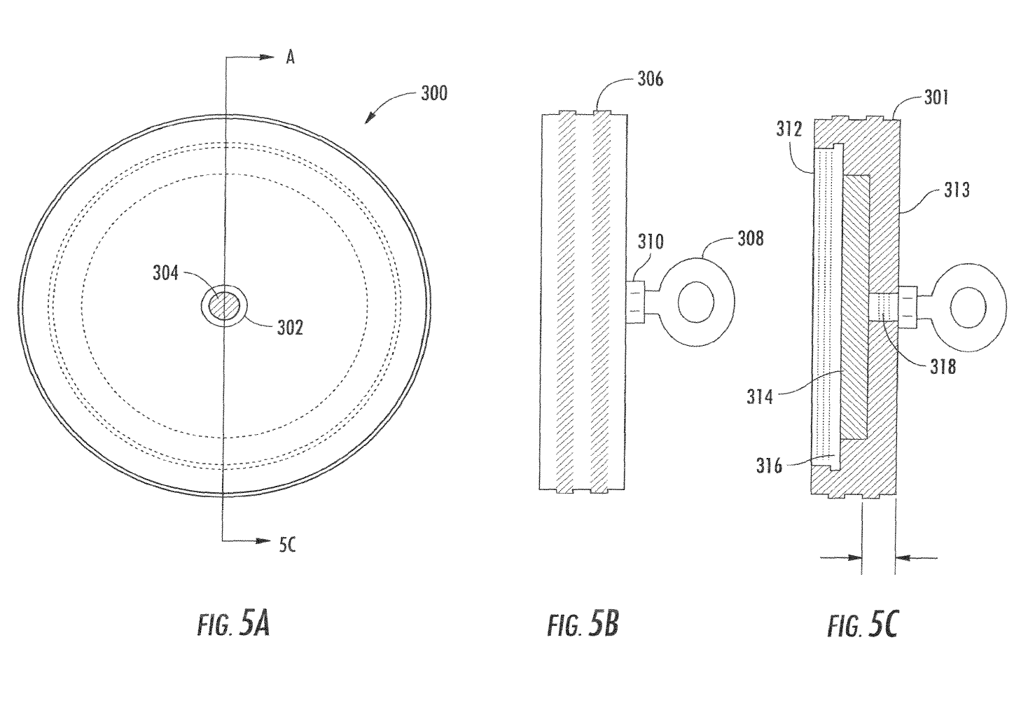

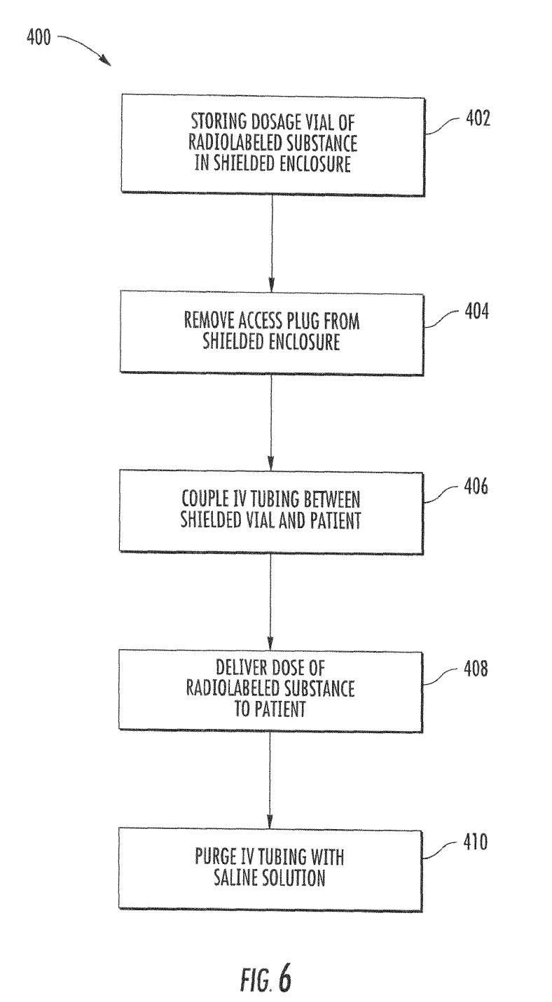

FIGS. 5A and 5B are top and side views, respectively, of the exemplary removable shielded panel or cover 300 shown in FIG. 2, and FIG. 5C is a sectional view along A-A of the embodiment of the removable radiation-shielded lid illustrated in FIG. 5A. The shielded cover 300 is sized and shaped to cover the relatively wide opening 204 (FIG. 3A) of the open-ended shielded vessel 202 (FIG. 3A), which is in turn sized and shaped to allow transfer of a patient dose vial 240 (FIG. 3A) into and out of the shielded interior region of the vessel 202.

In the exemplary embodiment, the shielded cover 300 is disk shaped, as in a jar lid. The shielded cover 300 includes an outer layer 301 the same type of shield material used in as the outer layer 224 (FIG. 3A) of the shielded vessel 202. The same or different materials can be used. A first cavity 312 is provided along a bottom surface of the shielded cover 300. The first cavity 312 is sized and shaped to form a relatively snug fit with an outer perimeter of the relatively wide opening 204 of the open-ended shielded vessel 202. In some embodiments, a side wall of the first cavity 312 includes one or more threads allowing a threaded engagement with a complementary thread 206 of the relatively wide opening 204.

The shielded cover 300 also includes a second cavity 313 extending away from the open end of the first cavity 312. The cavity is sized and shaped to accommodate a plug 314 or layer of the same shielding material as used for the inner layer 226 (FIG. 3A) of the shielded vessel 202. In the exemplary embodiment, the second cavity is disk-shaped to accommodate a disk 314 of Lexan® having approximately the same thickness as the inner layer 226 of the shielded vessel 202. The thickness of the shielded cover 300 adjacent to the Lexan disk 314 is at least as thick or thicker than the thickness of the outer layer 224 of the shielded vessel 202 to maintain shield uniformity around the entire shielded cavity when the shielded cover 300 is attached to the shielded vessel 202. In particular, the size and shape of the Lexan disk 314 is sufficient to cover the relatively wide opening 214 of the open-ended shielded vessel 202, for example having an outer diameter ID₂ (FIG. 3A).

In some embodiments, the shielded cover 300 includes an attachment element to facilitate hanging or otherwise supporting the vial shield during use. In the illustrative example, the shielded cover 300 includes an eyelet 308 centrally located along an outer, top surface of the shielded cover and extending away from the surface. The eyelet 308 may include a threaded shank 318 for fastening it to a threaded aperture 302 provided in the lid 301. A locking nut 310 may be included to further secure attachment of the eyelet 308 to the shielded cover 300.

FIG. 6 is a flow diagram of an embodiment of a process 400 for intravenously administering a radiolabeled substance. A dosage vial of radiolabeled substance is stored in shielded enclosure at 402. An access plug is removed from shielded enclosure at 404. IV tubing is coupled between shielded vial and patient at 406. A dose of radiolabeled substance is delivered to a patient at 408, and the IV tubing is purged with saline solution at 410.

After infusion is completed, the auxiliary IV set, the secondary IV set, and patient dose vial(s) should be disposed of appropriately. For example, these components should be returned to radiopharmacy such that any residual activity can be measured and recorded on a Case Report Form (CRF).

Onalta® (Y-90 Edotreotide), which is also know as 90Y-DOTA-tyr3-Octreotide, is administered by intravenous infusion to patients with refractory somatostatin-receptor positive tumors because of the large volume of the radiopharmaceutical therapy (86 mL or greater).

The Onalta® (Y-90 Edotreotide) infusion system (FIG. 1) allows for ease of administration of an Amino Acid solution and the Onalta® therapy through the same IV access site on the patient. The system has been designed to deliver the maximal amount of Onalta® therapy, while at the same time minimizing the radiation exposure to the staff through an innovative, proprietary Aluminum-Lexan Onalta® vial shield. The infusion system utilizes a standard dual-channel IV pump, which is commonly found in hospitals and clinics. All of the disposable infusion components used in the administration of Onalta® are standard, off-the-shelf components, which should be readily available in any hospital.

Although a dual channel infusion pump is described herein for infusing substances into a patient, other pumping means are envisioned, such as multiple single channel infusion pumps, gravity systems and combinations of any of these infusion pumping techniques.

Other embodiments will be evident to those of skill in the art. It should be understood that the foregoing detailed description is provided for clarity only and is merely exemplary. The spirit and scope of the present invention are not limited to the above examples, but are encompassed by the following claims.

What is claimed is:

1. A shielded enclosure suitable for reducing radiation exposure during infusion of a radioactive substance comprising:
a radiation shield defining a shielded cavity suitable for storing a vial containing at least one dose of a radioactive substance, the radiation shield further defining a first aperture providing external access to the shielded cavity and a second aperture suitable for transferring the vial into and out of the shielded cavity;
a shielded plug removably attachable to the radiation shield and adapted to shield the first aperture when attached thereto; and
a shielded panel removably attachable to the radiation shield and adapted to shield the second aperture when attached thereto, the radiation shield together with the shielded plug and the shielded panel when attached, forming a substantially continuous shielded cavity, the radiation shielding suitable for reducing radiation exposure during infusion of the radioactive substance from the vial to a patient.

2. The shielded enclosure of claim 1, wherein the radiation shield comprises a plurality of different shielding layers, the shielded plug and shielded panel, when attached to the radiation shield, preserving continuity the same plurality of different shielding layers about the substantially continuous shielded cavity.

3. The shielded enclosure of claim 2, wherein each of the plurality of different shielding layers is formed from a respective material selected from a group of materials consisting of: metals; aluminum; lead; steel; stainless steel; tungsten; titanium; metal alloys; leaded glass; polymers; polycarbonate materials; solids formed from synthetic resins; and wood.

4. The shielded enclosure of claim 2, wherein the radiation shield comprise an inner layer of polycarbonate material and an outer layer of metal.

5. The shielded enclosure of claim 4, wherein the metal is aluminum.

6. The shielded enclosure of claim 1, further comprising a attachment element allowing the shielded enclosure to be suspended from an intravenous (IV) pole.

7. The shielded enclosure of claim 1, further comprising a vial stored within the shielded cavity, the vial containing at least one dose of a radioactive substance, the vial including an access port substantially aligned with the first aperture when stored within the shielded cavity.

8. The shielded enclosure of claim 7, wherein the radioactive substance is a radioconjugate comprising an yttrium-90 radiolabeled somatostatin peptide or analog.

9. A method of administering a radiolabeled compound to a patient comprising:
placing a reservoir containing at least one dose of a radioactive compound in a shielded enclosure having a fluid access port;
providing a fluid communication channel between the reservoir and a patient;
delivering at least one dose of the radiolabeled compound through the fluid communication channel at a rate of about 500 mL/hour; and
washing the fluid communication channel after delivery of the radiolabeled compound, wherein radiation exposure during infusion of the radiolabeled compound into a patient is substantially reduced.

10. The method of claim 9, wherein the act of washing the fluid communication channel comprises flushing a saline solution through the fluid communication channel.

11. The method of claim 9, wherein the shielded enclosure comprises an interior polycarbonate layer and an exterior aluminum layer.

12. The method of claim 9, wherein the radiolabeled substance is yttrium-90 radiolabeled somatostatin peptide or analog.

13. The method of claim 9, further comprising delivering a non-radiolabeled compound through the fluid communication channel at a rate of about 500 mL/hour.

14. The method of claim 13, wherein delivery of the radiolabeled compound and the non-radiolabeled compound occur in succession.

15. An intravenous injection apparatus comprising:
a first reservoir storing a first non-radioactive compound;
a first fluid line in fluid communication between the first reservoir and a patient-side needle;
a second reservoir storing a saline solution;
a second fluid line in fluid communication with the patient-side needle; and
a vial shield surrounding a vial containing a radioactive compound, the vial in fluid communication with the second fluid line,
the apparatus operable to inject a dose of radioactive compound into a living subject operably coupled to the second end of the fluid line.

16. The intravenous injection apparatus of claim 15, wherein the vial shield comprises a substantially continuous aluminum shielding layer and a substantially continuous polycarbonate material shielding layer; the vial shield further comprising an access aperture providing access through the shielding layers.

17. The intravenous injection apparatus of claim 15, wherein the radioactive compound is a radioconjugate comprising an yttrium-90 radiolabeled somatostatin peptide or analog.

18. The intravenous injection apparatus of claim 15, wherein the non-radioactive compound comprises a diluted nutrient preparation containing amino acids.

19. The intravenous injection apparatus of claim 15 further comprising a dual channel infusion pump, a first channel of the pump adapted for infusing a fluid through the first fluid line and a second channel of the pump adapted for infusing a fluid through the second fluid line.

20. A method for reducing radiation exposure during infusion of a radioactive compound into a patient comprising:
storing a vial containing at least one dose of a radioactive compound in a shielded enclosure having an aperture blocked by a shielded access plug;
removing the shielded access plug from shielded enclosure thereby exposing the aperture;
coupling an intravenous (IV) fluid line between the vial containing the at least one dose of a radioactive compound and the patient, the coupling occurring through the exposed aperture;
infusing at least a portion of the at least one dose of a radioactive compound into the patient through the IV fluid line.

21. The method of claim 20, wherein the radioactive compound is a radioconjugate comprising an yttrium-90 radiolabeled somatostatin peptide or analog

22. The method of claim 20, further comprising infusing a non-radioactive compound into the patient through at least a patient proximal portion of the IV fluid line.

23. The method of claim 20, wherein the non-radioactive compound being a diluted nutrient preparation containing amino acids and radioactive compound being a radioconjugate comprising an yttrium-90 radiolabeled somatostatin peptide or analog are each infused alternately into the patient through at least a portion of the IV fluid line.

24. The method of claim 20, further comprising suspending the shielded enclosure containing at least one dose of a radioactive compound from an IV pole.
